(19) United States
(12) Patent Application Publication
(10) Pub. No.: US 2016/0243002 A1
(43) Pub. Date: Aug. 25, 2016

(54) MULTILAYER MINITABLETS WITH DIFFERENT RELEASE RATES

(71) Applicant: Apta Research, LLC, Pennsauken, NJ (US)

(72) Inventors: Siva Rama Krishna Nutalapati, Princeton, NJ (US); Rakeshkumar Khushalbhai Lad, Marlton, NJ (US)

(21) Appl. No.: 15/144,023

(22) Filed: May 2, 2016

Related U.S. Application Data
(63) Continuation of application No. 13/165,893, filed on Jun. 22, 2011, now abandoned.
(60) Provisional application No. 61/359,974, filed on Jun. 30, 2010.

(51) Int. Cl. A61J 3/10; A61K 31/7048; A61K 9/28; A61K 9/20; A61K 9/24; A61K 31/137; A61J 3/00

(52) U.S. Cl. CPC A61J 3/10; A61K 31/137; A61K 31/7048; A61J 3/005; A61K 9/2095; A61K 9/209; A61K 9/2866

(57) ABSTRACT

Multilayer minitablets for oral administration of a combination of active pharmaceutical ingredients which release the active pharmaceutical ingredient at different release rates are described.

This patent application is a continuation of U.S. application Ser. No. 13/165,893 filed Jun. 22, 2011, which claims the benefit of priority from U.S. Provisional Application Ser. No. 61/359,974, filed Jun. 30, 2010, teachings of each of which are hereby incorporated by reference in their entirety.

### FIELD OF THE INVENTION

The present invention relates to multilayer minitablets 5 mm or less in diameter for oral administration of a combination of active pharmaceutical ingredients. The minitablets are formulated so that the layers release the active pharmaceutical ingredients at different release rates. These minitablets are formulated into capsules for oral administration of the combination of active pharmaceutical ingredients.

### BACKGROUND OF THE INVENTION

A combination therapy for effecting weight loss and treating obesity with a sympathomimetic agent (e.g., phentermine or a phentermine-like drug) and an anticonvulsant sulfamate derivative (e.g. topiramate) is disclosed in U.S. Pat. Nos. 7,674,776; 7,659,256; 7,553,818; and 7,056,890. A disclosed preferred embodiment of pharmaceutical composition in these patents includes phentermine in an immediate release formulation and further includes topiramate in a controlled release formulation.

Published U.S. Application No. 2008/0113026 discloses a layered pharmaceutical formulation including two or more pharmaceutical layers and an intermediate layer disposed between the two or more pharmaceutical layers. A formulation wherein the first pharmaceutical layer comprises phentermine and the second pharmaceutical layer comprises topiramate is disclosed.

A delayed/extended release formulation of topiramate for once-a-day administration is disclosed in published U.S. Patent Application No. 2008/0085306.

A controlled release composition for treating obesity, diabetes or a related condition in a subject comprising topiramate, microcrystalline cellulose and methocellulose is disclosed in published U.S. Patent Application Nos. 2009/0304789 and 2009/0304785. The patent applications also disclose a method for treating obesity and/or effecting weight loss by administering topiramate and optionally, in addition, a sympathomimetic agent such as phentermine.

Published U.S. Patent Application No. 2006/0121112 also discloses a topiramate formulation with an immediate release component of topiramate and a delayed release component of topiramate.

### SUMMARY OF THE INVENTION

An aspect of the present invention relates to a multilayer minitablet 5 mm or less in diameter comprising at least two active pharmaceutical ingredients. The minitablets are formulated so that the different layers release the active pharmaceutical ingredients at different release rates.

In one embodiment, the multilayer minitablet comprises at least two active pharmaceutical ingredients useful in effecting weight loss or treating obesity.

In one embodiment, the multilayer minitablet of the present invention comprises one or more immediate release layer(s) containing as an active pharmaceutical ingredient an anti-obesity agent and one or more modified release layer(s) containing as an active pharmaceutical ingredient an anticonvulsant sulfamate derivative, compressed with or to the immediate release layer(s), to form a minitablet.

The minitablet may further comprise an optional inert layer or layers to control release of the active pharmaceutical ingredient from the modified release layer(s).

The minitablet may further comprise an optional functional or nonfunctional coating either partly or completely surrounding the minitablet.

The minitablet may further comprise an optional subcoating between the minitablet and the functional or nonfunctional coating.

Another aspect of the present invention relates to a capsule comprising a plurality of multilayer minitablets 5 mm or less in diameter, wherein each multilayer minitablet comprises at least two active pharmaceutical ingredients. The minitablets are formulated so that the different layers release the active pharmaceutical ingredients at different release rates.

In one embodiment, the multilayer minitablets of the capsule comprise at least two active pharmaceutical ingredients useful in effecting weight loss or treating obesity.

Another aspect of the present invention relates to methods for treating a subject with the capsules of the present invention.

In one embodiment, the present invention relates to a method for effecting weight loss or treating obesity in a subject which comprises orally administering to the subject a capsule comprising a plurality of multilayer minitablets 5 mm or less in diameter, wherein each multilayer minitablet comprises at least two active pharmaceutical ingredients useful in effecting weight loss or treating obesity and is formulated so that the different layers release the active pharmaceutical ingredients at different release rates.

### DESCRIPTION OF THE FIGURES

FIG. 1 is a diagram showing an embodiment of a multilayer minitablet of the present invention. In this embodiment, the minitablet comprises one or more immediate release layer(s) compressed with one or more modified release layer(s). The minitablet may further comprise optional inert layers to control release of the active pharmaceutical ingredient from the modified release layer(s).

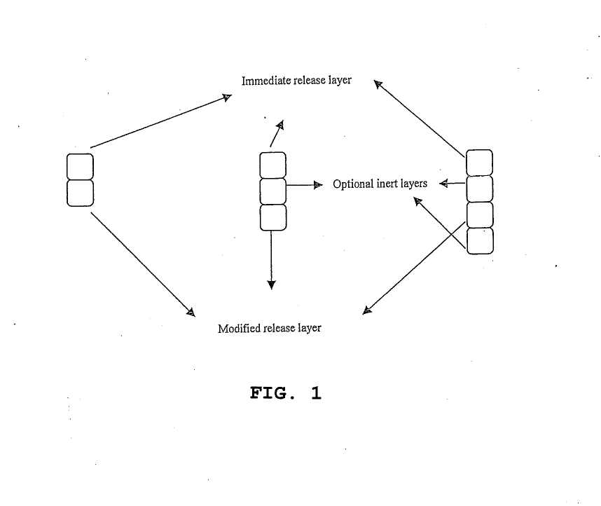

FIG. 2 is a diagram of an embodiment of a functionally coated multilayer minitablet of the present invention.

### DETAILED DESCRIPTION OF THE INVENTION

The present invention relates to multilayer minitablets 5 mm or less in diameter for oral administration of a combination of active pharmaceutical ingredients. The minitablets are formulated so that the different layers release the active pharmaceutical ingredients at different release rates.

Any active pharmaceutical ingredient administered orally can be formulated in accordance with the present invention as these multilayer minitablets provide a particularly useful formulation to achieve simultaneous release of two or more different drugs and different release rates from a single dosage form.

In one embodiment of the present invention, the active pharmaceutical ingredient of the minitablets in the immediate release layer is an anti-obesity agent. In one embodiment of the present invention, the anti-obesity agent is phentermine, sibutramine, pramlintide, olanzapine, amantadine, bupropion and/or salts thereof.

In one embodiment of the present invention, the active pharmaceutical ingredient of the minitablets in the modified release layer is an anticonvulsant agent. In one embodiment of the present invention, the anticonvulsant agent is topiramate, Zonisamide and/or a salt thereof.

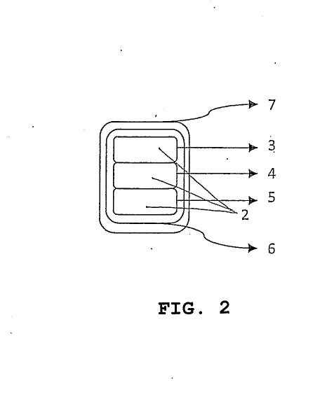

In one embodiment, as depicted in FIG. 2, the multilayer minitablet of the present invention comprises a minitablet 2 with one or more immediate release layer(s) 3 containing an active pharmaceutical ingredient such as an anti-obesity agent and one or more modified release layer(s) 5 containing an active pharmaceutical ingredient such as an anticonvulsant agent compressed with or to the immediate release layer(s) to form a minitablet. The minitablet may further comprise an optional inert layer 4 in between the immediate release layer (s) 3 and the modified release layer(s) 5 to control release of the active pharmaceutical ingredient from the modified release layer(s). The minitablet may further comprise a functional coating or a non-functional coating 7 and an optional subcoating 6 in between the minitablet 2 and the functional coating or non-functional coating 7.

In one embodiment of the present invention, the multilayer minitablet comprises a bilayer minitablet with an immediate release layer containing phentermine and/or a salt thereof or a phentermine-like drug and a modified release layer containing topiramate and/or a salt thereof compressed with or to the immediate release layer.

In one embodiment, the bilayer minitablet is coated with a functional coating or non-functional coating around the entire surface of the bilayer minitablet. In this embodiment, the minitablet may further comprise an optional subcoating between the minitablet and the functional coating or non-functional coating. In this embodiment, the minitablet may comprise a functional coating and a non-functional coating. In this embodiment, the minitablet may further comprise optional inert layers to control release of the active pharmaceutical ingredient from the modified release layer(s). By “inert layer” it is meant a layer containing no drug or drug in an amount which is insignificant to the therapeutic activity of the minitablet or formulations thereof.

As will be understood by the skilled artisan upon reading this disclosure, additional immediate release layers and/or modified release layers containing an active pharmaceutical ingredient and/or additional inert layers can be added to the minitablet, and minitablets comprising these additional layers are encompassed by the present invention. Addition of such layers in no way circumvents the present invention.

Immediate release layer(s) as used herein means that part of the minitablet that releases substantially all of the active pharmaceutical agent contained therein in 120 minutes in a suitable in vitro dissolution test. A suitable exemplary dissolution test may be, for example, dissolution carried out in 900 mL of phosphate buffer (pH 6.8) at a temperature of 37.0° C.±0.5° C. using USP apparatus I (basket) rotating at a speed of 100 rpm. However, as will be understood by the skilled artisan upon reading this disclosure, variations on this test as well as the apparatus and conditions well known to those skilled in the art can be used.

Various methods for preparation of immediate release layers and the vehicles therein are well-known in the art. Generally recognized compendiums of such methods and ingredients include Remington: The Science and Practice of Pharmacy, Alfonso R. Gennaro, editor, 20th ed. Lippincott Williams & Wilkins: Philadelphia, Pa., 2000 and Sheth et al. Compressed Tablets, in Pharmaceutical Dosage Forms: Tablets, Vol 1. edited by H. A. Lieberman and L. Lachman, Dekker N.Y. (1980).

The immediate release layer or layers of a minitablet of the present invention can be prepared by direct compression of a mixture of the active pharmaceutical ingredient with a suitable carrier or excipient, such as carbohydrate or protein fillers, such as sugars, including lactose, sucrose, mannitol, or sorbitol; starch from corn, wheat, rice, potato, or other plants; celluloses, such as methyl cellulose, hydroxypropyl methyl-cellulose, sodium carboxymethylcellulose, or microcrystalline cellulose; gums including arabic and tragacanth; proteins such as gelatin and collagen; inorganics, such as kaolin, calcium carbonate, dicalcium phosphate, sodium chloride; magnesium carbonate; magnesium oxide; and other agents such as acacia and alginic acid.

Agents that facilitate disintegration and/or solubilization can also be added, such as cross-linked polyvinyl pyrrolidone, sodium starch glycolate, croscarmellose sodium, alginic acid, or a salt thereof, such as sodium alginate, microcrystalline cellulose and corn starch.

Tablet binders that can be used include acacia, methylcellulose, sodium carboxymethylcellulose, polyvinylpyrrolidone (povidone), hydroxypropyl cellulose, hydroxypropyl methylcellulose, sucrose, starch and ethylcellulose.

Lubricants that can be used include magnesium stearates, stearic acid, sodium Stearyl fumerate, talc, waxes, oils, silicon dioxide and colloidal silica.

Fillers, agents that facilitate disintegration and/or solubilization, tablet binders and lubricants, including the aforementioned, can be used singly or in combination.

The immediate release layer or layers of the minitablets are formulated, for example, by preparing a powder or granular mixture by dry blending, slugging or dry or wet granulating, optionally adding a disintegrant and/or a lubricant and compressing into minitablet layers.

Modified release layer(s) as used herein means that part of the minitablet having a dissolution profile that is extended, delayed or controlled as compared to the 120 minute in vitro dissolution of the immediate release layer(s). In one embodiment, the modified release layer has a release profile of 4 hours or greater.

Various methods for preparation of modified release layers and the vehicles therein are well-known in the art. Generally recognized compendiums of such methods and ingredients include Remington: The Science and Practice of Pharmacy, Alfonso R. Gennaro, editor, 20th ed. Lippincott Williams & Wilkins: Philadelphia, Pa., 2000 and Sheth et al. Compressed Tablets, in Pharmaceutical Dosage Forms: Tablets, Vol 1. edited by H. A. Lieberman and L. Lachman, Dekker N.Y. (1980).

A modified release layer or layers of a minitablet of the present invention can be prepared by incorporating release retarding excipients into the above-described formulation for the immediate release layer(s), and/or by either completely omitting or reducing the amount of disintegrants in the above-described formulation for the immediate release layer(s).

Examples of release retarding excipients include, but are not limited to, hydrophilic polymers such as hydroxypropylmethylcellulose, hydroxymethylcellulose, hydroxypropylcellulose and hydroxyethylcellulose, and which swell in contact with aqueous liquids, and control release of the drug by diffusion through the swollen polymer network.

Examples of other release retarding excipients include, but are not limited to, waxes such as carnauba wax, bees wax stearic acid and gums such as acacia, acrylic polymers, shellac, zein, polyvinylpyrrolidine including crosslinked polyvinylpyrrolidinone, vinyl acetate copolymers, polyethylene oxides, polyvinyl alcohols, and combinations comprising at least one of the foregoing materials.

Enteric polymers can also be used as release retarding excipients to modify the release rates in certain pH environments. Examples of enteric polymers include, but are not limited to, polymers such as methacrylic acid-ethyl acrylate copolymer (1:1), ethacrylic acid-methyl methacrylate copolymer (1:1), methacrylic acid-methyl methacrylate copolymer (1:2), polyvinyl acetate phthalate (PVAP), hydroxypropyl methylcellulose acetate succinate (HPMCAS) and cellulose acetate phthalate (CAP).

The modified release layer or layers of the minitablets are formulated, for example, by preparing a powder or granular mixture of drug or drugs with release retarding excipients and/or other acceptable pharmaceutical excipients by dry blending, slugging or dry or wet granulating, optionally adding a lubricant and compressing the mixture into tablet layers.

The optional inert layer or layers may comprise any biocompatible compound or mixture of compounds. The inert layer may be soluble or insoluble, permeable or impermeable, pH dependent or pH independent or any combination thereof depending upon the drug or drugs to be orally administered and/or the release mechanism required. Preferably, the inert layers are inert, insoluble and impermeable to drug contained in the modified release layer(s). Accordingly, the inert layer preferably comprises no drug or drug in an amount which is insignificant to the therapeutic activity of the minitablet or formulations thereof.

Exemplary biocompatible materials for use in the inert layer include, but are not limited to, waxes, polymers, gums and other pharmaceutically acceptable excipients either alone or in combination.

Exemplary wax excipients include, but are not limited to, wax and wax-like excipients such as carnauba wax, vegetable wax, fruit wax, microcrystalline wax, bees wax (white or bleached, and yellow), hydrocarbon wax, paraffin wax, cetyl esters wax or a combination comprising at least one of the foregoing waxes. Other suitable wax excipients include, for example, fatty alcohols (such as lauryl, myristyl, stearyl, cetyl or specifically cetostearyl alcohol), hydrogenated vegetable oil, hydrogenated castor oil, fatty acids such as stearic acid, fatty acid esters including fatty acid glycerides (mono-, di-, and tri-glycerides), polyethylene glycol (PEG) having a molecular weight of greater than about 3000 number average molecular weight, Mₙ (e.g. PEG 3350, PEG 4000, PEG 4600, PEG 6000, and PEG 8000), or a combination comprising at least one of the foregoing.

Exemplary polymer excipients include, for example acrylic polymers, alkylcelluloses including substituted alkylcelluloses, shellac, zein, polyvinylpyrrolidine including crosslinked polyvinylpyrrolidinone, vinyl acetate copolymers, polyethylene oxides, polyvinyl alcohols, and combinations comprising at least one of the foregoing materials.

Suitable acrylic polymers that can be used in the inert layer include, but are not limited to, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, or a combination comprising at least one of the foregoing polymers.

Suitable alkylcelluloses and substituted alkyl celluloses include, but are not limited to, methyl cellulose, ethylcellulose, hydroxy or carboxy substituted alkyl celluloses (e.g., hydroxyl propylcellulose, crosslinked hydroxypropylcellulose, carboxymethylcellulose, crosslinked sodium carboxymethylcellulose), hydroxy substituted alkyl-alkyl celluloses (e.g., hydroxypropylmethylcellulose), or a combination comprising at least one of the foregoing.

Exemplary additional pharmaceutically acceptable excipients for use in the inert layer include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, magnesium aluminum silicate (Veegum), and larch arabogalactan); alginates; polyethylene oxide; inorganic calcium salts; silicic acid; and combinations thereof.

Fillers, tablet binders and lubricants, including the aforementioned, can be used in the inert layer singly or in combination.

The inert layer of the minitablet of the present invention is formulated, for example, by preparing a powder or granular mixture of one or more pharmaceutically acceptable excipients by dry blending, slugging or dry or wet granulating, optionally adding a lubricant and compressing the mixture into minitablet layers.

One or more of the immediate release layers and one or more of the modified release layers and optionally, one or more of the inert layers are then compressed together to form a single minitablet of the present invention.

In one embodiment, the entire minitablet is then coated with a functional coating or a non-functional coating. By “functional coating” it is meant a coating or film that further modifies the release properties of the formulation. Examples of such functional coatings or films include, but are not limited to, controlled release, delayed release, sustained release, modified release, pH dependent, pH independent coatings, and any combinations thereof. By “non-functional coating” it is meant a coating that does not significantly modify the release properties of the total formulation, for example, a cosmetic coating.

In one embodiment, the minitablet is coated with a functional coating and a nonfunctional coating.

In one embodiment, the functional coating of the minitablet of the present invention is porous, thereby permitting quick release of the active pharmaceutical ingredient from the immediate release layer upon oral administration of the minitablet. In one embodiment, the functional coating also works synergistically with the matrix of the modified release layer to control the release of the active pharmaceutical ingredient from the modified release layer of the minitablet. In one embodiment, the minitablet of the present invention is coated with more than one functional coating.

The functional coating material can be in the form of a film coating comprising a solution or dispersion or a compressible powder mixture of a hydrophilic or hydrophobic polymer. Solvents used for application of the functional coating include pharmaceutically acceptable solvents, such as water, methanol, ethanol, methylene chloride, and a combination comprising at least one of the foregoing solvents.

Examples of functional coating materials include, but are not limited to, film forming polymers such as an alkylcellulose including methylcellulose or ethylcellulose, a hydroxyalkylcellulose such as hydroxymethylcellulose, hydroxyethylcellulose, hydroxypropylcellulose and hydroxybutylcellulose, a hydroxyalkyl alkylcellulose such as hydroxyethyl methylcellulose and hydroxypropyl methylcellulose, a carboxyalkylcellulose such as carboxymethylcellulose, an alkali metal salt of carboxyalkylcelluloses such as sodium carboxymethylcellulose, a carboxyalkyl alkylcellulose such as carboxymethyl ethylcellulose, a carboxyalkylcellulose ester, a starch, a pectin such as sodium carboxymethylamylopectine, a chitin derivate such as chitosan, a polysaccharide such as alginic acid, alkali metal and ammonium salts thereof, a carrageenan, a galactomannan, tragacanth, agar-agar, gum arabicum, guar gum and xanthan gum, acrylic acid, polyacrylic acid and the salts thereof, a polyvinylalcohol, a polyvinylpyrrolidone, a copolymer of polyvinylpyrrolidone with vinyl acetate, a polyalkylene oxide such as polyethylene oxide and polypropylene oxide and a copolymer of ethylene oxide and propylene oxide, or a combination comprising at least one of the foregoing.

The functional coating may optionally comprise a plasticizer, an additional film-former, a pore former, and/or a combination comprising at least one of the foregoing.

Examples of enteric polymers include, but are not limited to, polymers such as methacrylic acid-ethyl acrylate copolymer (1:1), ethacrylic acid-methyl methacrylate copolymer (1:1), methacrylic acid-methyl methacrylate copolymer (1:2), polyvinyl acetate phthalate (PVAP), hydroxypropyl methylcellulose acetate succinate (HPMCAS) and cellulose acetate phthalate (CAP). Additionally, dyestuffs or pigments can be added to the enteric polymer coating for product identification or to characterize the quantity of active compound, i.e., dosage. Further, the enteric polymer can be optionally modified to include a pore-forming agent thereby resulting in a semi-enteric coating.

The pore-forming agents used are preferably water soluble materials. Examples of pore-forming materials include, but are not limited to, polymers like hydroxyalkyl celluloses such as hydroxymethyl cellulose, hydroxyethyl cellulose, hydroxypropyl cellulose and hydroxybutylcellulose, hydroxyalkyl alkylcelluloses such as hydroxyethyl methylcellulose and hydroxypropyl methylcellulose, polyvinylalcohols, polyvinylpyrrolidones, copolymers of polyvinylpyrrolidone with vinyl acetate, sugars, salts and combinations thereof.

In some embodiments, prior to applying the functional coating, the minitablet is coated with a subcoating or non-functional coating and then coated with the functional coating to avoid interactions of the active pharmaceutical ingredients with the functional coating. Such a subcoating may serve as an interlayer coating used to separate a functional coating from other components of the formulation. Examples of subcoating materials include, but are not limited to, film forming polymers like hydroxyalkyl celluloses such as hydroxymethyl cellulose, hydroxyethyl cellulose, hydroxypropyl cellulose and hydroxybutylcellulose, hydroxyalkyl alkylcelluloses such as hydroxyethyl methylcellulose and hydroxypropyl methylcellulose, polyvinylalcohols, polyvinylpyrrolidones, copolymers of polyvinylpyrrolidone with vinyl acetate, and combinations thereof.

In some embodiments, these minitablets of the present invention are further coated with a non-functional coating.

A plurality of minitablets of the present invention are then encapsulated into a capsule for oral administration to a subject for effecting weight loss or treating obesity.

In one embodiment, the capsule is ingested intact.

In another embodiment, the capsule is opened and the minitablets therein are sprinkled over a soft food such as apple sauce, custard, ice cream, oatmeal, pudding or yogurt to facilitate swallowing. In this embodiment, it is preferred that the minitablets be sprinkled over no more than a teaspoonful of food.

Capsules of the present invention are preferably administered once a day.

As will be understood by the skilled artisan upon reading this disclosure, while the above relates to formulations for weight loss and anti-obesity, any orally active pharmaceutical ingredient can be included as a drug in the multilayer minitablets of the present invention. Examples include, but are not limited to, alpha-2 adrenergic agents, analgesics, angiotensin-converting enzyme (ACE) inhibitors, antianxiety agents, antiarrhythmics, antibacterials, antibiotics, anticoagulants, anticonvulsants, antidepressants, antidiabetics, antiemetics, antiepileptics, antifungals, antihelminthics, antihistamines, antihyperlipidemics, antihypertensives, antiinfectives, antimalarials, antimicrobials, antimigraine agents, antimuscarinic agents, antineoplastic agents, antiprotozoals, antipsychotics, antispasmodics, antiretroviral agents, antivirals, attention-deficit hyperactivity disorder (ADHD) agents, β-blockers, calcium channel blockers, chemotherapeutic agents, cholinesterase inhibitors, Cox-2 inhibitors, decongestants, diuretics, histamine-2 receptor antagonists, hypnotics, hypoglycemic agents, hypotensive agents, immunosuppressants, lipotropics, neuroleptics, opioid analgesics, peripheral vasodilators/vasoconstrictors, proton pump inhibitors, sedatives, serotonin receptor agonists, sympathomimetics as well as pharmaceutically acceptable salts, solvates, hydrates, stereoisomers (racemates, individual enantiomers or diastereomers, or any combination thereof), or polymorphs thereof, or pharmaceutically acceptable combinations comprising at least one of the foregoing active agents, and the like.

The following nonlimiting examples are provided to further illustrate the present invention.

### EXAMPLES

### Example 1

Preparation of Immediate release Phentermine HCl (37.5 mg) and Modified release Topiramate (200 mg) Multilayer Tablets

The immediate release drug containing layer contained phentermine HCl (37.50 mg/capsule), lactose monohydrate (46.50 mg/capsule), hydroxypropyl cellulose (10.00 mg/capsule), croscarmellose sodium (5.00 mg/capsule) and magnesium stearate (1.00 mg/capsule).

The modified release drug containing layer contained topiramate (200.00 mg/capsule), carnauba wax (142.00 mg/capsule), stearic acid (50.00 mg/capsule), silicon dioxide (4.00 mg/capsule) and magnesium stearate (4.00 mg/capsule).

The inert layer contained carnauba wax (50.00 mg/capsule), dibasic calcium phosphate (29.00 mg/capsule), stearic acid (20 mg/capsule) and magnesium stearate (1.00 mg/capsule).

The subcoating contained Opadry II Clear (24 mg/capsule) and purified water which was removed during processing.

The extended release functional coating contained Surelease (37.50 mg/capsule), hydroxypropyl methyl cellulose (37.50 mg/capsule) and purified water which was removed during processing.

The immediate release drug containing layer of the minitablet was prepared as follows:

phentermine HCl was dry blended with all the ingredients except croscarmellose sodium and magnesium stearate and the blend was granulated with purified water. The granulate was dried and milled through a suitable screen. Croscarmellose sodium and magnesium stearate were screened and then added to the milled granules. The mixture was then blended for about 2 minutes.

The modified release drug containing layer of the minitablet was prepared as follows:

topiramate and carnauba wax were mixed and granulated with a solution of stearic acid in ethyl alcohol. The granulate was then dried and milled through a suitable screen. Silicon dioxide and magnesium stearate were screened and then added to the milled granules. The mixture was then blended for another 2 minutes.

The inert layer was prepared as follows:

carnauba wax and dicalcium phosphate were mixed and granulated with a solution of stearic acid in ethyl alcohol. The granulate was then dried and milled through a suitable screen. Magnesium stearate was screened and then added to the milled granules. The mixture was then blended for another 2 minutes.

The immediate release drug containing layer, modified release drug containing layer and inert layer blends were then compressed into a multilayer minitablet of 15 mg each in the following sequence using a multilayer tablet press: 10 mg of the modified release drug containing layer, 2.5 mg of the inert layer and 2.5 mg of the immediate release drug containing layer. Minitablets were then subcoated.

The subcoating was prepared by dissolving Opadry II Clear in purified water and spraying the solution onto the multilayer minitablets in a Wurster equipped fluid bed apparatus.

The modified release coating was prepared as follows: in a container, purified water was mixed with hydroxypropyl methyl cellulose using a mixer until all of the hydroxypropyl methyl cellulose was completely dissolved. The hydroxypropyl methyl cellulose solution was then added to a Surelease dispersion and mixed for 15 minutes. The resulting dispersion was continuously mixed throughout the entire coating process. Using Wurster equipped fluid bed apparatus, the Surelease/hydroxypropyl methyl cellulose dispersion was sprayed onto the subcoated tablets until the required weight gain was achieved.

What is claimed is:

1: A method for producing a multilayer minitablet 5 mm or less in diameter, said method comprising:
preparing a first powder or granular mixture comprising a first active pharmaceutical ingredient and a suitable carrier or excipient by dry blending, slugging or dry or wet granulating;
preparing a second powder or granular mixture comprising a second active pharmaceutical ingredient, a suitable carrier or excipient and release retarding agent by dry blending, slugging or dry or wet granulating;
adding the second powder or granular mixture to a multilayer tablet press;
adding the first powder or granular mixture to the multilayer tablet press; and
compressing the first powder or granular mixture and the second powder or granular mixture together to form a single minitablet comprising an immediate release layer and a modified release layer; and
partially or completely coating the minitablet with a functional coating comprising at least 50% of a pore-forming agent which permits quick release of the first active pharmaceutical ingredient from the immediate release layer while also working with the modified release layer to control the release of the active pharmaceutical ingredient from the modified release layer of the minitablet.

2: The method of claim 1 further comprising:
preparing a third inert powder or granular mixture comprising one or more pharmaceutically acceptable excipients by dry blending, slugging or dry or wet granulating;
adding the third powder or granular mixture to the multilayer tablet press; and
compressing the first, second and third powders or granular mixtures together to form a single minitablet comprising an immediate release layer, an inert layer, and a modified release layer or an immediate release layer, a modified release layer and an inert layer.

3: The method of claim 2 wherein the inert layer is inert, insoluble and impermeable to the second pharmaceutical ingredient contained in the modified release layer and controls release of the second active pharmaceutical ingredient from said modified release layer.

4: The method of claim 1 further comprising applying a subcoating between the multilayer minitablet and the extended release functional coating.

5: The method of claim 1 further comprising completely surrounding the multilayer minitablet with an outer non-functional coating.

6: The method of claim 1 wherein the functional coating comprises a solution or dispersion of a hydrophilic or hydrophobic polymer in a pharmaceutically acceptable solvent.
